(19) United States
(12) Patent Application Publication
(10) Pub. No.: US 2009/0226389 A1
(43) Pub. Date: Sep. 10, 2009

(54) OXIDANT FOR HAIR TREATMENT

(75) Inventors: Jonathan Frank Warr, Paris (FR); Emmanuel Julien Aussant, Paris (FR); Stuart Bernard Fraser, Neston (GB)

Correspondence Address: SUGHRUE-265550, 2100 PENNSYLVANIA AVE. NW, WASHINGTON, DC, 20037-3213, US

(73) Assignee: TAKASAGO INTERNATIONAL CORPORATION, Tokyo (JP)

(21) Appl. No.: 12/280,533

(22) PCT Filed: Feb. 22, 2007
(86) PCT No.: PCT/EP07/51718

§ 371 Date: Jan. 16, 2009

(30) Foreign Application Priority Data
Feb. 22, 2006 (FR) 0650611

(51) Int. Cl. A61K 8/22; A61K 33/40; A61K 33/04; A61K 8/24; A61Q 5/08; A61Q 5/00

(52) U.S. Cl. 424/62; 424/613; 424/709; 424/616; 8/406; 424/70.6

(57) ABSTRACT

The invention relates to an aqueous oxidant for hair treatment which comprises the following, by weight based on the total weight of the oxidant: 1) 3 to 15% of a peroxide and/or a persulfate; 2) 0.05 to 2% of at least one perfume containing: •5 to 100%, by weight based on the weight of the perfume, of olfactory substances having at least one ester group, and •0 to 95% by weight based on the weight of the perfume, of olfactory substances having at least one alpha, β-unsaturated ketone group, said olfactory substances being stable in the presence of peroxide; 3) 0.1 to 5% of a solubilizer; and 4) 0.005 to 2% of a sequestering agent. Application: cosmetic composition for coloring or bleaching the hair.

The present invention relates to cosmetics and more particularly to hair treatment.

The different approaches to hair bleaching or coloring are described in the book entitled “Hair and Hair Care”, D. H. Johnson, ISBN 0-8247-9365-X.

Hair bleaching has long been carried out using aqueous oxidizing compositions comprising an oxidant based on peroxide and/or persulfate and an oxidant activator.

The most commonly used activator is ammonia, which accelerates the destruction of the melamine in keratin fibers and increases the rate of oxidation of these fibers. Furthermore, ammonia makes it possible to adjust the pH of the oxidizing compositions, which must have an alkaline pH in order to be active.

There are three categories of compositions for hair coloring: compositions for temporary hair coloring, compositions for semipermanent hair coloring and compositions for permanent hair coloring.

The compositions for temporary hair coloring generally comprise water-soluble acidic colorants and water-soluble pigments, which are deposited on the surface of the hair. These colorants are removed simply by shampooing.

The compositions for semipermanent hair coloring contain nitroaniline derivatives, nitrophenylenediamine derivatives and nitroaminophenol derivatives as colorants. These colorants penetrate the cuticle and partially penetrate the cortex of the hair. The coloring effects of these colorants can withstand 5-10 shampooings.

Two distinct compositions are used for permanent hair coloring. One of the compositions contains coloring precursors, e.g. phenylenediamine, 2,5-diaminotoluene, N,N-bis(2-hydroxymethyl)-p-phenylenediamine and p-aminophenol, and couplers, e.g. resorcinol, chlororesorcinol, methylresorcinol, alpha-naphthol, m-aminophenol and m-phenylenediamine, in an alkaline agent.

The other composition is a stabilized solution of hydrogen peroxide.

These two compositions are mixed just before use. The precursors and the hydrogen peroxide diffuse into the sheath of the hair, where the coloring takes place after a series of chemical reactions.

The coloring precursors are oxidized by the hydrogen peroxide to form p-benzoquinone-imines or -diimines, for example, which are reactive intermediates in the formation of the coloration.

The couplers, which are relatively stable in the presence of hydrogen peroxide, react rapidly with the intermediates to form colorant molecules. These molecules are too large to escape from the structure of the hair each time it is shampooed. Nevertheless, because the hair grows, it has to be recolored regularly.

The compositions for permanent hair coloring are generally called oxidizing hair colorants.

In these compositions the hydrogen peroxide also serves as bleaching agent for the hair's natural pigment.

The composition of coloring precursors generally contains ammonia.

Unfortunately, ammonia releases a very unpleasant, pungent odor when these compositions are used, which is disturbing for the user as well as for the hairdresser and the other people present in the hairdressing salon, house or bathroom.

Several solutions have been proposed for avoiding or masking this unpleasant odor.

For example, hair coloring compositions having an acidic or neutral pH have been proposed (cf. international patent applications WO 98/27943, WO 98/27944 and WO 98/27945).

It has also been suggested to follow the hair bleaching compositions containing ammonia with perfumed conditioners, which release a strong odor to mask the ammonia.

A composition containing geometrical isomers of trans-2,2,6-trimethylcyclohexylcarboxylate which may mask the bad odors, notably those of coloring agents containing ammonia is described in EP 1 347 035 A2.

It has also been proposed to replace the ammonia with:
other alkaline agents such as ethanolamine, guadinium salts, basic amino acids, polyamines, hydroxides, etc. (cf., for example, U.S. Pat. No. 4,507,278 and EP 0 512 879);
dicarbonyl compounds (cf. U.S. Pat. No. 3,816,615);
alkali metal hydroxides (cf. U.S. Pat. No. 4,226,852).

It has also been proposed to reduce the concentration of ammonia and use a water-soluble thickening surfactant (U.S. Pat. No. 4,507,278).

Most of these alternative solutions have not been used or are less efficient than ammonia (at least in some cases) since they have other disadvantages, such as the shorter duration of the coloration or the change in coloration over time.

Consequently, the great majority of commercial hair bleaching or coloring compositions contain ammonia and it is advisable to find solutions for masking its odor.

The problem associated with the odor of ammonia is not specific to hair coloring or bleaching. It arises e.g. in waste treatment, the environment, animals, or the developing of diazotized substances.

Various solutions have been proposed in these fields, such as the use of perfumes (patents FR 2 197 973, U.S. Pat. No. 2,546,791, WO 02/041927, JP 2003/010300, JP 60/185,560, JP 2001/161799), by-products of vanilla manufacture (U.S. Pat. No. 4,551,305), inhibitors or complexing agents (patent EP 1 214 878).

Apart from perfumes, the solutions proposed in these fields cannot be carried over to hair coloring or bleaching. It is actually essential for the ammonia to remain free in order to produce the expected effect, so it is not possible to use the inhibitors or complexing agents proposed in other fields.

Perfumes have already been used to mask the odor of ammonia generated when hair is treated with ammonia-based compositions.

For example, patent application EP 1 190 696 describes a cosmetic composition for the hair which contains cis-hex-3-en-1-ol, an alkaline agent such as ammonia, and an aromatic alcohol. Such a composition can be used to prepare hair bleaching or coloring compositions.

Perfumes such as geraniol, cis-hex-3-en-1-ol, linalool, alpha-terpinol, beta-damascone, alpha-damascone, beta-ionone, etc. have been described as being capable of masking the ammonia produced by certain cosmetic products containing it (patent KR 2000/025753).

Patent application JP 2000/344629 describes deodorants for masking the odor of ammonia released by hair perming preparations. These perfumes are selected from anethole, aurantiol, dimetol, cyclohexyl butylate, estragol, lavandulol, methyl-γ-ionone, para-cresol and para-cresol acetate.

Patent EP 1 133 982 describes ammonia-based cosmetic compositions for hair bleaching or coloring which comprise a perfume that is olfactorily stable in ammonia and:

1) either contains a phenyl ring and has a diffusion coefficient in air of more than 5.7;

2) or contains a C₅ ring including at least one sp2 hybridized carbon atom and has a diffusion coefficient in air of more than 4.4.

In all the proposed solutions, the perfume is used in combination with the ammonia because it is widely accepted in this field that the peroxide used as oxidant degrades the perfume much more than the ammonia used as alkaline substance.

EP patent application 1 346 720 A2 relates to compositions useful for making the unpleasant odors of ammonia of coloring or bleaching hair compositions.

These compositions contain at least a perfume or an essential oil which is stable in an acid medium as well as in an alkaline medium.

It has now been found, surprisingly, that, in hair coloring or bleaching compositions, the perfume can be added to the oxidant, making it possible to use olfactory substances which are not stable in the alkaline substance.

Advantageously, it is now possible to combine the perfume present in the ammonia with a second perfume present in the oxidant.

Contrary to the existing prejudices against the introduction of a perfume into the oxidant, it is possible according to the invention to:

1) create a very wide variety of perfume notes when using hair coloring or bleaching products; and

2) avoid unwanted chemical reactions before said products are used.

Thus the present invention relates primarily to an aqueous oxidant (bleaching agent) for hair treatment which comprises the following by weight based on the total weight of the oxidant:

1) 3 to 15% of a peroxide and/or a persulfate;

2) 0.05 to 2% of a perfume containing:
5 to 100%, preferably 10-96% and particularly preferably 15-84%, by weight based on the weight of the perfume, of olfactory substances having at least one ester group, and
0 to 95%, preferably 4-90% and particularly preferably 16-85%, by weight based on the weight of the perfume, of olfactory substances having at least one α,β-unsaturated ketone group,

said olfactory substances being stable in the presence of peroxide and unstable in the presence of ammonia;

3) 0.1 to 5% of a solubilizer; and

4) 0.005 to 2% of a sequestering agent.

The present invention further relates to a hair bleaching composition which comprises the following, separately packaged:

1) an oxidant as defined above; and

2) an alkaline activator optionally containing a perfume.

The invention further relates to hair coloring compositions which comprise the following, separately packaged:

1) an oxidant as defined above; and

2) an alkaline colorant containing coloring precursors, couplers and optionally a perfume.

Advantageously, the alkaline activators and colorants in the above compositions comprise ammonia, ethanolamine, aminomethylpropanol or mixtures thereof and preferably ammonia. They can also contain at least one perfume of which the constituent olfactory substances must be stable in ammonia.

The stability of the olfactory substances in the presence of a peroxide or ammonia can advantageously be determined by the method of extraction with CO₂ (carbon dioxide) in the supercritical phase, followed by GC-MS analysis (gas chromatography and gas spectrometry) (J. Rowe, Chemistry and Technology of Flavors and Fragrances, CRC Press, ISBN 1-4051-1450-9).

This extraction and analysis method consists in determining the initial concentration of the olfactory substance in an aqueous composition containing 6% by weight of peroxide or 2.3% by weight of ammonia, and the concentration at a given time. Appropriate aqueous compositions for these tests are described below. The olfactory substance must be perfectly solubilized and the resulting product stable.

For the purposes of the present invention, an olfactory substance stable in the presence of peroxide is understood as meaning an olfactory substance whose initial concentration between 0.05 to 2% by weight in a 6% by weight aqueous solution of peroxide, e.g. hydrogen peroxide, has not been reduced by more than 50% after four weeks of storage in the dark at 40° C. in a closed glass vessel, and that the weight concentration of peroxide, e.g. hydrogen peroxide, has not dropped by more than 20% relative to a control without olfactory substance.

The concentration of peroxide is determined by the standard analytical method using the reduction of potassium permanganate (KMnO₄) by hydrogen peroxide in sulfuric acid.

Likewise, an olfactory substance stable in the presence of ammonia is understood as meaning an olfactory substance whose initial concentration between 0.05 to 2% by weight in a 2.3% by weight aqueous solution of ammonia has not been reduced by more than 50% after four weeks of storage in the dark at 40° C. in a closed glass vessel.

The perfumes used according to the invention consist of conventional olfactory substances and solvents that are commonly used in this field, such as isopropyl glycol, dipropylene glycol, isopropyl myristate, triethyl citrate, benzyl benzoate, dialkyl adipates, diethyl phthalate or propylene glycol.

Examples of olfactory substances containing at least one ester group are acetates such as phenylethyl acetate or octyl acetate, prenyl acetate (CAS 1191-16-8), cis-hex-3-enyl acetate (CAS 3681-71-8), propionates such as phenylethyl propionate (CAS 122-70-3), ethylbutyrate (CAS 105-54-4), isobutyrates such as phenoxyethyl isobutyrate (CAS 103-60-6), and more complex esters such as hedione (CAS 24851-98-7), lactones such as gamma-decalactone (CAS 706-14-9), gamma-undecalactone (CAS 104-67-6) or gamma-nonalactone (CAS 104-61-0), coumarin (CAS 91-64-5), and musks, including macrocyclic musks such as musk T (CAS 105-93-3) and musk C14 (CAS 54982-83-1), which are also both lactones.

Olfactory substances containing at least one α,β-unsaturated ketone group are preferably alicyclic α,β-unsaturated ketones. Examples of olfactory substances containing an α,β-unsaturated ketone group are the ketones alpha-damascone (CAS 24720-09-0), beta-damascone (CAS 23726-92-3), delta-damascone (CAS 57378-68-4) and damascenone (CAS 23696-85-7), ionones such as beta-ionone (CAS 014901-07-6), and irones such as alpha-irone (CAS 79-69-6).

In the present description, diketones which have a resonance structure with α, β-unsaturated ketones, e.g. cyclotene (CAS 000080-71-7) and hinokitiol (CAS 000499-44-5), are also referred to as α,β-unsaturated ketones.

The other constituents of the perfumes are olfactory substances selected from alcohols, nitrites, ketones, ethers and essential oils, are preferably stable in peroxide, and may be stable or unstable in ammonia. Examples of such substances which may be mentioned are alcohols such as linalool (CAS 78-70-6) and phenylethyl alcohol (CAS 60-12-8), ethers, e.g. diphenyl ether (CAS 101-84-8), dibenzyl ether (CAS 103-50-4) and galaxolide (CAS 1222-05-5), nitrites, e.g. geranyl nitrile (CAS 5146-66-7), and ketones, e.g. ambretone (CAS 37609-25-9), fenchone (CAS 1195-79-5) and phenyl ketones such as celestolide (CAS 13171-00-1) and benzylacetone (CAS 2550-26-7).

Preferably, the perfume used in the oxidant will be also stable in the presence of peroxide. For the purpose of the invention, a stable perfume is a perfume whose initial weight concentration has not been reduced by more than 50%, preferably by more than 25% under the same conditions as those defined hereinabove for the olfactory substance.

Examples of olfactory substances for perfumes that are particularly appropriate for the purposes of the invention are listed in Table I below.

TABLE I
Examples of olfactory substances for perfumes
that are stable in the presence of a peroxide

| Olfactory substance | CAS | Stability/peroxide |
|---|---|---|
| Musk C14 | 54982-83-1 | Yes |
| Cis-hex-3-en-1-yl acetate | 3681-71-8 | Yes |
| Methyl salicylate | 119-36-8 | Yes |
| Gamma-undecalactone | 104-67-6 | Yes |
| Delta-dodecalactone | 713-95-1 | Yes |
| Perilla aldehyde | 2111-75-3 | Yes |

Examples of olfactory substances stable in the presence of ammonia that are suitable for the purposes of the invention are alcohols, nitrites, certain ketones, ethers, musks not containing an ester group, and olfactory substances having bulky or highly branched ester groups, such as isoamyl isobutyrate or givescone (CAS 57934-97-1).

Examples of such olfactory substances that are particularly appropriate for the purposes of the invention are listed in Table II below.

TABLE II

| Olfactory substance | CAS | Stability/ammonia |
|---|---|---|
| Linalool | 78-70-6 | Yes |
| Floralzone | 67634-15-5 | Yes |
| Phenylethyl alcohol | 60-12-8 | Yes |
| Diphenyl ether | 101-84-8 | Yes |
| Ambretone | 37609-25-9 | Yes |
| Citronellyl butanoate | 141-16-2 | Yes |

The peroxide used in the oxidant according to the invention can be selected from any of the peroxides commonly used in hair bleaching compositions. Examples of particularly appropriate peroxides which may be mentioned are hydrogen peroxide and hydrogen persulfate, hydrogen peroxide being very particularly preferred.

The amount of peroxide to be used is generally between 3% and 15% by weight, based on the total weight of the oxidant, and is often about 6%.

In the present description the percentage of peroxide is the percentage of pure peroxide and not the percentage of commercially available peroxide solution.

The amount of perfume to be used in the oxidant according to the invention must be sufficient to mask the odor of ammonia when it is mixed with the activator or the colorant (with or without a perfume in the activator or the colorant), in the case where said agent is used in a hair coloring or bleaching composition.

The amounts of perfume in the oxidants according to the invention are between about 0.05% and 2% by weight based on the weight of the total composition of the oxidant.

Amounts below 1% and more typically below 0.5% by weight, based on the weight of the total composition of the oxidant, are generally sufficient.

The solubilizer in the oxidant is a compound which favors the solubilization of the perfume in the aqueous peroxide solution and which is stable under acidic conditions.

These solubilizers (and/or emulsifiers) are surfactants that are commonly used in cosmetic products and can be non-ionic, anionic, cationic or amphoteric solubilizers or a mixture of several surfactants of identical type. Examples of appropriate surfactants for the purposes of the invention are described in the book entitled “Cosmetic Bench Reference” (2005), ISBN 1-932633-10-3.

Furthermore, the oxidant according to the invention can contain a solvent and a thickener which are stable in acidic media.

Examples of solvents and thickeners, as well as surfactants, are described in U.S. Pat. No. 6,887,280, which is incorporated in the present description by reference.

The metal sequestering agent used in the oxidant according to the invention is a compound that is commonly used for sequestering metals in solution.

Examples of appropriate sequestering agents are given in the book entitled “Cosmetic Bench Reference (2005), ISBN 1-932633-10-3”. The following compounds are very particularly preferred: sodium EDTA, sodium triphosphate, phosphoric acid, sodium phosphonates (particularly those marketed under the trade name DEQUEST® by Monsanto Corp.), sodium stannate, tetrasodium phosphate, biodegradable tetrasodium glutamate diacetate (marketed by Alzo under the trade name “dissolvine GL38”) or mixtures thereof. The total concentration of sequestering agent is normally between 0.005 and 2% and preferably less than 0.5%.

The oxidant according to the invention can also contain other ingredients commonly used in this field, such as foaming additives, antifoaming additives, additives for giving volume to the hair, and agents for improving the visual appearance of the hair or the cosmetic composition.

Bleaching Cosmetic Compositions

The hair bleaching cosmetic compositions according to the invention comprise:

1) an oxidant as defined above; and

2) an alkaline activator containing ammonia and optionally a perfume.

Advantageously, the weight ratio of oxidant to activator is about 1:5 to 5:1.

Coloring Cosmetic Compositions

The hair coloring cosmetic compositions according to the invention comprise:

1) an oxidant as defined above; and

2) an alkaline colorant containing coloring precursors, couplers and optionally a perfume.

Advantageously, the weight ratio of oxidant to colorant is about 1:5 to 5:1.

The ammonia-based colorant typically contains:
an alkaline agent such as ammonia, ethanolamine, aminomethylpropanol or mixtures thereof;
buffers such as oleic acid, dilinoleic acid, ethanolamine hydrochloride, ammonium chloride, ammonium acetate, citric acid, lactic acid, sulfuric acid, lauric acid and tetrasodium pyrophosphate;
surfactants;
solvents;
thickeners;
antioxidants and metal chelating agents such as ascorbic acid, erythorbic acid, thiolactic acid and mixtures thereof;
coloring precursors and couplers; and
esthetic additives or additives for improving appearance.

For further details, reference may be made to the book entitled Hair and Hair Care, published by D. H. Johnson, ISBN 0-8247-9365-X.

The coloring precursors can be any of those commonly used for hair coloring. Several examples are given in CBR, Cosmetic Bench Reference (2005), ISBN 1-932633-10-3, in the section entitled “Hair Colorant”. It is also possible to use the hydroxyethyl-p-phenylenediamine sulfate (CAS no. 93841-25-9) marketed by Wella.

The couplers can be selected e.g. from resorcinol, chlororesorcinol, methyl-resorcinol, alpha-naphthol, m-aminophenol and m-phenylenediamine.

Other coloring precursors and couplers appropriate for the purposes of the invention appear in annexes III and IV of Directive 76/768/EEC relating to cosmetic agents. These annexes are regularly updated on the basis of the opinion of the Scientific Committee on Cosmetic Products and Non-Food Products (SCCNFP), which has recently been replaced by the Scientific Committee on Consumer Products (SCCP). For example, the nomenclature of cosmetic products was reviewed in 2000 and the opinion expressed by the abovementioned Committee (SCCNFP/0299/00) allowed updating of the list of ingredients used in cosmetics of Directive 96/335/EEC. A summary of the recent adaptations for technical advance can be found in the report SCCNFP/0098/99 (from page 84 onwards) and a recent updating of annex IV on the basis of the opinion of the SCCNFP is contained in Directive 2005/42/EC.

It is also possible to use the coloring precursors and couplers listed in Commission Directive 96/335/EEC, which is periodically updated by the SCCNFP, now called the SCCP.

Another possibility is to use the coloring precursors and couplers that appear in the list drawn up by the International Nomenclature Committee of the Association of Cosmetics, Toilet Products and Perfumes. This list appears in the International Cosmetic Ingredient Dictionary, 10th edition, 2004, AISN 1882621344.

The invention also related to a hair bleaching kit which contains the following, separately packaged:
a hair cosmetic bleaching according to the invention and optionally at least one of the following ingredients:
a shampoo;
an after-shampoo;
a perfume and an oil for conditioning the hair after bleaching.

The invention also related to a hair coloring kit which contains the following, separately packaged:
a hair coloring cosmetic composition according to the invention and optionally at least one of the following ingredients:
a shampoo;
an after-shampoo;
a perfume and an oil for conditioning the hair after coloring.

The invention will now be illustrated by the following non-limiting Examples.

### EXAMPLES

The oxidant and colorant below were used in the following Examples in which the parts are parts by weight and the percentages are percentages by weight on the basis of the total weight of the oxidant or colorant.

### The Oxidant:

Hydrogen peroxide; 20 parts of a 30% solution of active agent (VWR International, 201 rue Carnot, 94126 Fontenay-sous-Bois, France)
Water; 78.6 parts
EDTA; 0.1 part (EdetaR powder, BX BASF)
Phosphoric acid; 0.3 part of a 10% solution of active agent (BASF)
Marlipal 24/99 (laureth C12-C14); 1% part (Sasol)
Perfume; up to 0.5% part
Adjustment of pH to 3±0.2 with phosphoric acid

The Colorant:
Water; QS
EDTA; 0.2%
Sorbitol; 1.25% (Lonza)
Polyquaternium 22; 1.0% (Merck)
Sodium metasilicate; 0.02% (Lipo Chemicals Inc.)
Steareth-21; 3.0% (Uniquema)
Dilinoleic acid; 5.0% (Cognis)
Oleth-10; 6.0% (Croda)
Cocamide MEA; 5.0% (Cognis)
Lineoleamidopropyl dimethylamine dimer disodium; 4.0% (Alzo)
Incroquat behenyl; 0.9% (Croda)
Ammonium hydroxide; 8% of a 28% solution of active agent (VWR)
Sodium sulfite; 0.3% (VWR)
Sodium sulfate; 0.35% (VWR)
Perfume; up to 0.5%
Jarocol* TDS: 0.66%-2-methyl-p-phenylenediamine sulfate
Jarocol* RL: 0.3%-1,3-benzenediol
Jarocol* MAP: 0.03%-m-aminophenol
   Jarocol: products marketed by James Robinson Ltd, Huddersfield, UK

### Example 1

### Floral Note

The perfume having the composition below (Perfume 1) was added at a rate of 0.3% by weight to the oxidant defined above. This gave an oxidant which could advantageously be used with the colorant defined above to mask the unpleasant odor of ammonia.

### Perfume 1:

| Olfactory substance | CAS | % by weight |
|---|---|---|
| Gamma-undecalactone | 104-67-6 | 6.9 |
| Gamma-nonalactone | 104-61-0 | 1 |
| Damascenone | 23696-85-7 | 1 |
| Beta-damascone | 23726-91-3 | 3 |
| Gamma-decalactone | 706-14-9 | 3.6 |
| Dipropylene glycol | 25265-71-8 | 33.6 |
| Methyldioxolan | 6413-10-1 | 1.3 |
| Hedione | 24851-98-7 | 36 |
| Beta-ionone | 79-77-6 | 2 |
| Musk T | 105-95-3 | 3 |
| Phenoxyethyl isobutyrate | 103-60-6 | 1.6 |
| Santalex T | 68877-29-2 | 7 |

### Example 2

### Apple Note

The qualities of apple notes created with:
either a single perfume, i.e. 0.5% of Perfume 2 in the colorant, mixed with an equal amount of oxidant without perfume;
or two perfumes, i.e. 0.25% of Perfume 2 in the colorant, mixed with an equal amount of oxidant containing 0.25% of Perfume 3, were compared.

It was found that, by virtue of the substances stable in the oxidant but not stable in the colorant, the result with two perfumes was superior.

### Perfume 2:

| Olfactory substance | CAS | Parts by weight |
|---|---|---|
| C-6 alcohol | 111-27-3 | 1 |
| Citronellyl nitrile | 51566-62-2 | 2 |
| Dimethylbenzylcarbinyl | 151-05-3 | 5 |
| acetate | | |
| Dynascone | 56973-85-4 | 0.1 |
| Thesaron | 22471-55-2 | 1 |
| Ethyl 2-methylbutyrate | 7452-79-1 | 4 |
| Fleuramone | 137-03-1 | 1 |
| Cis-hex-3-en-1-ol | 928-96-1 | 5 |
| Isocyclocitral | 1335-66-6 | 1 |
| Iso E Super | 54464-57-2 | 7 |
| Linalool | 78-70-6 | 8 |
| Manzanate | 39255-32-8 | 7 |
| Menthol | 2216-51-5 | 1.5 |
| Sandolore | 65113-99-7 | 2 |
| Verdox | 88-41-5 | 25 |

### Perfume 3:

| Olfactory substance | CAS | Parts by weight |
|---|---|---|
| Gamma-undecalactone | 104-67-6 | 30 |
| Ethyl butyrate | 105-54-4 | 10 |
| Musk T-93 | 105-95-3 | 10 |
| Prenyl acetate | 1191-16-8 | 16 |
| Cis-hex-3-enyl acetate | 3681-71-8 | 2 |
| Beta-ionone | 79-77-6 | 1 |

### Example 3

### Citrus Note

The qualities of citrus notes created with:
either a single perfume, i.e. 0.5% of Perfume 4 in the colorant, mixed with an equal amount of oxidant without perfume,
or two perfumes, i.e. 0.1% of Perfume 4 in the colorant, mixed with an equal amount of oxidant containing 0.4% of Perfume 5, were compared.

It was found that, by virtue of the substances stable in the oxidant but not stable in the colorant, the result with two perfumes was superior.

### Perfume 4:

| Olfactory substance | CAS | Parts by weight |
|---|---|---|
| Hexanol | 111-27-3 | 1 |
| Citronellyl nitrile | 51566-62-2 | 2 |
| 9-Decen-1-ol | 13019-22-2 | 0.5 |
| Dynascone | 56973-85-4 | 0.3 |
| Cineole | 470-82-6 | 2 |
| Cis-hex-3-en-1-ol | 928-96-1 | 3 |
| Isocyclocitral | 1335-66-6 | 1 |
| 2-Isopropyl-4-methylthiazole | 15679-13-7 | 0.01 |
| Linalool | 78-70-6 | 10 |
| Menthol | 2216-51-5 | 1 |
| Grapefruit acetal | 67647-46-8 | 5 |
| Orange oil terpenes white | 68647-72-3 | 5 |
| Raspberry ketone | 5471-51-2 | 1 |
| Rhubafuran | 82461-14-1 | 0.1 |
| Verdox | 88-41-5 | 10 |

### Perfume 5:

| Olfactory substance | CAS | Parts by weight |
|---|---|---|
| C-14-aldehyde | 104-67-6 | 7 |
| Citronellyl nitrile | 51566-62-2 | 2 |
| Hedione | 24851-98-7 | 5 |
| Cis-hex-3-enyl acetate | 3681-71-8 | 5 |
| Musk T-93 | 105-95-3 | 10 |
| Spaermint oil | 8008-79-5 | 2 |
| Tridecene-2-nitrile | 22629-49-8 | 1 |

### Example 4

The following perfumes are examples of perfumes useful for the present invention which may be used in the oxidant.

### Perfume 6:

| Olfactory substance | CAS | Parts by weight |
|---|---|---|
| Gamma-undecalactone gamma | 104-67-6 | 7 |
| Benzyl acetone | 2550-26-7 | 2.5 |
| Butyl acetate | 123-86-4 | 10 |
| Beta damascone | 23726-92-3 | 0.2 |
| Ethyl acetate | 141-78-6 | 1 |
| Cis-hex-3-enyl acetate | 3681-71-8 | 0.4 |
| Beta Ionone | 014901-07-6 | 1 |
| Verdox | 88-41-5 | 7 |

### Perfume 7:

| Olfactory substance | CAS | Parts by weight |
|---|---|---|
| Benzyl acetone | 2550-26-7 | 3 |
| Damascenone | 23696-85-7 | 0.5 |
| Beta-dihydro ionone | 17283-81-7 | 1.5 |
| Ethyl 2-methyl butyrate | 7452-79-1 | 1.5 |
| Manzanate | 39255-32-88 | 2 |
| Verdox | 88-41-5 | 7 |

### Perfume 8:

| Olfactory substance | CAS | Parts by weight |
|---|---|---|
| Gamma-nonalactone | 104-61-0 | 2 |
| Dimethyl benzyl carbinyl butyrare | 10094-34-5 | 3 |
| Ethyl acetate | 141-78-6 | 40 |
| Habonolide | 34902-57-3 | 4 |
| Phenyl ethyl propionate | 122-70-3 | 6 |
| Tridecene 2 nitrile | 22629-49-8 | 0.5 |

1. An aqueous oxidant for hair treatment which comprises the following by weight based on the total weight of the oxidant:
1) 3 to 15% of a peroxide and/or a persulfate;
2) 0.05 to 2% of at least one perfume containing:
5 to 100% by weight based on the weight of the perfume, of olfactory substances having at least one ester group, and
0 to 95% by weight based on the weight of the perfume, of olfactory substances having at least one α,β-unsaturated ketone group, said olfactory substances being stable in the presence of peroxide and unstable in the presence of ammonia;
3) 0.1 to 5% of a solubilizer; and
4) 0.005 to 2% of a sequestering agent.

2. The oxidant according to claim 1 wherein the peroxide is hydrogen peroxide.

3. The oxidant according to claim 1 wherein the persulfate is hydrogen persulfate.

4. The oxidant according to claim 1 wherein the perfume contains:
10 to 96%, by weight based on the weight of the perfume, of olfactory substances having at least one ester group, and/or
4 to 90%, by weight based on the weight of the perfume, of olfactory substances having at least one α,β-unsaturated ketone group.

5. The oxidant according to claim 1 wherein the perfume contains:
15 to 84%, by weight based on the weight of the perfume, of olfactory substances having at least one ester group, and/or
16 to 85%, by weight based on the weight of the perfume, of olfactory substances having at least one α,β-unsaturated ketone group.

6. The oxidant according to claim 1 to wherein the olfactory substances having at least one ester group are acetates, propionates or isobutyrates.

7. The oxidant according to claim 1 wherein the olfactory substances having at least one α,β-unsaturated ketone group are alicyclic ketones.

8. The oxidant according to claim 1, wherein the olfactory substances are selected among phenylethyl acetate, octyl acetate, prenyl acetate, cis-hex-3-enyl acetate, phenyl ethyl propionate, phenoxy ethyl isobutyrate, ethyl butyrate, hedione, gamma-decalactone, gamma-undecalactone, gamma-nonalactone, coumarin, Musk T, Musk C-14, alpha-damascone, beta-damascone, delta-damascone, damascenone, beta-ionone, alpha-irone, cyclotene and hinokitiol.

9. A cosmetic composition for bleaching the hair which comprises the following, separately packaged:
1) an oxidant as defined in claim 1; and
2) an alkaline activator optionally containing a perfume.

10. The cosmetic composition according to claim 9 wherein the alkaline activator comprises ammonia, ethanolamine, aminomethylpropanol or mixtures thereof.

11. The composition according to claim 9 wherein the weight ratio of oxidant to activator is between about 1:5 and 5:1.

12. A cosmetic composition for coloring the hair which comprises the following, separately packaged:
1) an oxidant as defined in claim 1; and
2) an alkaline colorant containing coloring precursors, couplers and optionally a perfume.

13. The cosmetic composition according to claim 12 wherein the weight ratio of oxidant to colorant is between about 1:5 and 5:1.

14. The cosmetic composition according to claim 12 wherein the alkaline colorant contains ammonia, ethanolamine, aminomethylpropanol or mixtures thereof.

15. A hair bleaching kit which contains the following, separately packaged:
a cosmetic composition according to any one of claims 9 to 11 and optionally at least one of the following ingredients:
a shampoo;
an after-shampoo;
a perfume and an oil for conditioning the hair after bleaching.

16. A hair coloring kit which contains the following, separately packaged:
a cosmetic composition according to claim 12 and optionally at least one of the following ingredients:
a shampoo;
an after-shampoo;
a perfume and an oil for conditioning the hair after coloring.
